(19) United States
(12) Patent Application Publication
(10) Pub. No.: US 2013/0123501 A1
(43) Pub. Date: May 16, 2013

(54) PROCESS FOR THE PREPARATION OF THE COMPOUND OSI-906

(71) Applicants: Arlindo L. Castelhano, New City, NY (US); Gary A. Cutting, Dorset (GB); Andrew J. Locke, Delmar, NY (US); Kristen Michelle Mulvihill, Dix Hills, NY (US); Robert Norrie, Selkirk, NY (US); Andrew J. O'brien, Albany, NY (US); Stuart R. Park, Dorset (GB); Josef A. Rechka, Port Jefferson, NY (US); Andrew MIchael Stevens, Delmar, NY (US); Christopher I. Thomas, Dorset (GB)

(72) Inventors: Arlindo L. Castelhano, New City, NY (US); Gary A. Cutting, Dorset (GB); Andrew J. Locke, Delmar, NY (US); Kristen Michelle Mulvihill, Dix Hills, NY (US); Robert Norrie, Selkirk, NY (US); Andrew J. O'brien, Albany, NY (US); Stuart R. Park, Dorset (GB); Josef A. Rechka, Port Jefferson, NY (US); Andrew MIchael Stevens, Delmar, NY (US); Christopher I. Thomas, Dorset (GB)

(21) Appl. No.: 13/812,629

(22) PCT Filed: Jul. 29, 2011
(86) PCT No.: PCT/US2011/045807

§ 371 Date: Jan. 28, 2013

Related U.S. Application Data
(60) Provisional application No. 61/369,132, filed on Jul. 30, 2010.

(51) Int. Cl. C07D 487/04

(52) U.S. Cl. CPC C07D 487/04
USPC 544/350

(57) ABSTRACT

Process for preparing the tyrosine kinase inhibitor OSI-906 comprises coupling Compound (2) with Compound (6) under specified conditions.

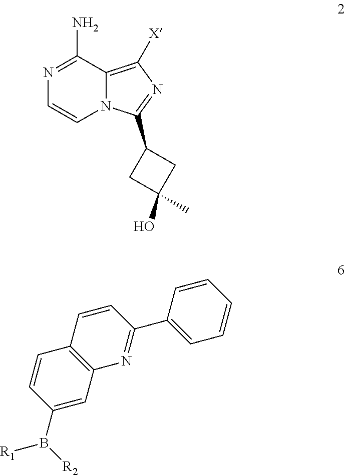

This application claims priority of U.S. Appl. No. 61/369,132, filed Jul. 30, 2010, the content of which is incorporated herein in its entirety by this reference.

### FIELD AND BACKGROUND

The present invention relates to chemical synthetic processes, and related intermediates and products, compositions, and uses thereof.

The development of target-based anti-cancer therapies has become the focus of a large number of pharmaceutical research and development programs. Various strategies of intervention include targeting protein tyrosine kinases, including receptor tyrosine kinases believed to drive or mediate tumor growth.

Insulin-like growth factor-1 receptor (IGF-1R) is a receptor tyrosine kinase that plays a key role in tumor cell proliferation and apoptosis inhibition, and has become an attractive cancer therapy target. IGF-1R is involved in the establishment and maintenance of cellular transformation, is frequently overexpressed by human tumors, and activation or overexpression thereof mediates aspects of the malignant phenotype. IGF-1R activation increases invasion and metastasis propensity.

Inhibition of receptor activation has been an attractive method having the potential to block IGF-mediated signal transduction. Anti-IGF-1R antibodies to block the extracellular ligand-binding portion of the receptor and small molecules to target the enzyme activity of the tyrosine kinase domain have been developed. See Expert Opin. Ther. Patents, 17(1):25-35 (2007); Expert Opin. Ther. Targets, 12(5):589-603 (2008); and Am J. Transl. Res., 1:101-114 (2009).

US 2006/0235031 (published Oct. 19, 2006) describes a class of bicyclic ring substituted protein kinase inhibitors, including Example 31 thereof, which corresponds to the dual IR/IGF-1R inhibitor known as OSI-906. As of 2011, OSI-906 is in clinical development in various cancers and tumor types. The preparation and characterization of OSI-906, which can be named as cis-3-[8-amino-1-(2-phenyl-quinolin-7-yl)-imidazo[1,5-a]pyrazin-3-yl]-1-methylcyclobutanol, is described in the aforementioned US 2006/0235031.

OSI-906 is a potent, selective, and orally bioavailable dual IGF-1R/IR kinase inhibitor with favorable drug-like properties. The selectivity profile of OSI-906 in conjunction with its ability to inhibit both IGF-1R and IR affords the special opportunity to fully target the IGF-1R/IR axis. See Future Med. Chem., 1(6), 1153-1171, (2009).

It is desirable to develop novel processes to prepare imidazopyrazine compounds, namely OSI-906, which may be practical, economical, efficient, reproducible, large scale, and meet regulatory requirements.

### SUMMARY OF THE INVENTION

The present invention is directed to chemical synthetic processes for preparing OSI-906 and various salts thereof. The invention includes the associated intermediates and salts of OSI-906. The invention further includes compositions of OSI-906 prepared according to the invention and uses thereof.

Therefore, in some aspects, the invention includes a method of preparing OSI-906 (Compound 1):

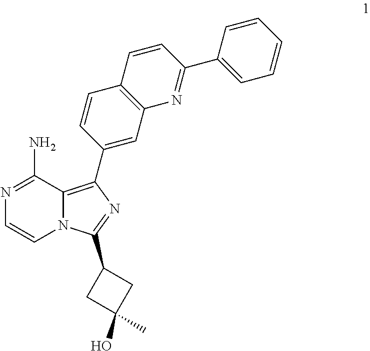 [SMILES: C[C@]1(O)C[C@@H](c2nc(-c3ccc4ccc(-c5ccccc5)nc4c3)c3c(N)nccn32)C1]

and pharmaceutically acceptable salts thereof, preferably at production scale, high yield and high purity comprising coupling Compounds (2) and (3).

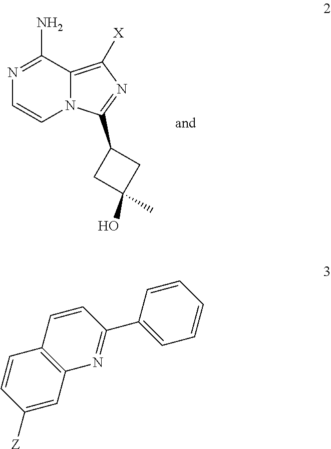

wherein Z is defined below.

In some embodiments, Compound (2) can be prepared by aminating Compound (4) under certain conditions.

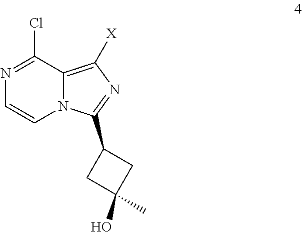

In some embodiments, Compound (4) can be prepared by methylating Compound (5) under certain conditions.

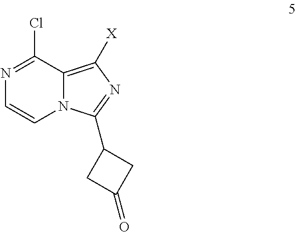

### BRIEF DESCRIPTION OF THE DRAWINGS

FIG. 1: DSC thermogram of the hydrochloride salt of OSI-906.

FIG. 2: XRPD pattern of the hydrochloride salt of OSI-906.

FIG. 3: ¹H NMR spectrum (in DMSO-d₆) of the hydrochloride salt of OSI-906.

FIG. 4: DSC thermogram of the tartrate salt of OSI-906.

FIG. 5: XRPD pattern of the tartrate salt of OSI-906.

FIG. 6: ¹H NMR spectrum (in DMSO-d₆) of the tartrate salt of OSI-906.

FIG. 7: DSC thermogram of the sulfate salt of OSI-906.

FIG. 8: XRPD pattern of the sulfate salt of OSI-906.

FIG. 9: ¹H NMR spectrum (in DMSO-d₆) of the sulfate salt of OSI-906.

FIG. 10: DSC thermogram of the fumarate salt of OSI-906.

FIG. 11: XRPD pattern of the fumarate salt of OSI-906.

FIG. 12: ¹H NMR spectrum (in DMSO-d₆) of the fumarate salt of OSI-906.

FIG. 13: DSC thermogram of the mesylate salt of OSI-906.

FIG. 14: XRPD pattern of the mesylate salt of OSI-906.

FIG. 15: ¹H NMR spectrum (in DMSO-d₆) of the mesylate salt of OSI-906.

### DETAILED DESCRIPTION OF THE INVENTION

In some embodiments, the present invention concerns a method of preparing OSI-906 (1) and salts thereof, by reacting (2) and (3), and isolating and purifying the product to afford at least about 1 kg of OSI-906;

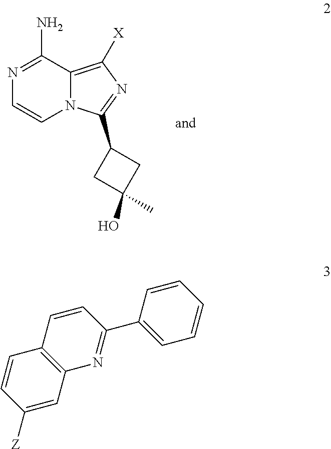

wherein X is Cl, Br or I and Z is —MgCl or a boronic ester/acid, ZnCl, SiEt₂Cl, SnR¹ wherein R¹ is C₁-C₆aliphatic.

In some embodiments, the process comprises
(a) reacting

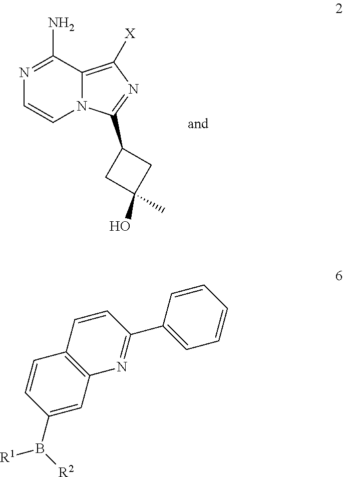

under palladium catalyst mediated coupling conditions;
wherein X is Cl, Br, or I; R² and R³ are independently selected from OH or OR⁴; or R² and R³ combine to form a cyclic boronic ester; and R⁴ is C₁-C₆ aliphatic;
(b) diluting with water and collecting solids.

In some embodiments, the process further comprises:
(c) suspending the solids in water and adjusting the pH to about 2-3 with a suitable acid to provide a dissolved salt form of OSI-906;
(d) separating remaining solids from liquid;
(e) mixing the liquid from (d) with a suitable metal scavenging resin to remove palladium;
(f) removing the resin;
(g) diluting the liquid with a suitable alcohol;
(h) adjusting the pH with base to about 5-6 to precipitate the OSI-906;
(i) isolating the precipitate from (h).

In some embodiments, there is thereby obtained at least about 1 kg of OSI-906 without further purification, and having a purity of at least about 90%.

In some embodiments, reaction (a) can be carried out in DMF, water DMA, NMP, toluene, acetonitrile, dioxane, DME, THF, 2-propanol, methanol, ethanol, n-propanol, n-butanol, sec-butanol, t-butanol and iso-butanol with cesium or sodium carbonate, potassium carbonate, potassium phosphate, potassium or sodium hydroxide, thallium hydroxide, thallium carbonate, barium hydroxide, silver oxide, cesium fluoride, tetrabutylammonium fluoride, tetraalkylammonium hydroxides, or alkyl amines.

The present invention further concerns reaction (a), which can be carried out in DMF with cesium carbonate or sodium carbonate.

The present invention further concerns the acid in (c), which can comprise hydrochloric acid.

Furthermore, isolation conditions were developed to address issues associated with large scale purification challenges. Namely, a work up sequence for the Suzuki coupling reaction to remove unwanted impurities (8), (9) and palladium without the use of column chromatography or more laborious purification techniques was developed. Not to be bound by theory, the purification conditions were identified that exploit the water solubility of the hydrochloride salt of (1) and poor water solubility of (8) at specifically tailored pH, respectively. The purification conditions exploit the water solubility of (9) and poor water solubility thereof at a different specifically chosen pH.

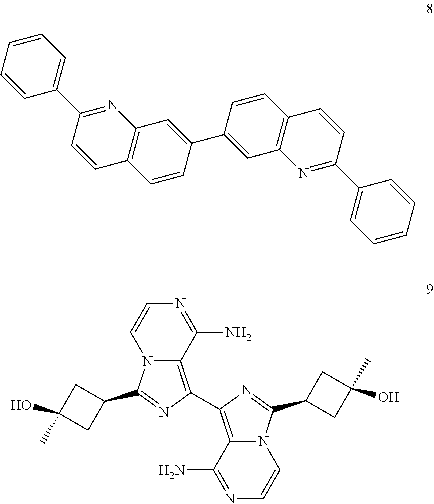 [SMILES: c1ccc(-c2ccc3ccc(-c4ccc5ccc(-c6ccccc6)nc5c4)cc3n2)cc1 ; C[C@]1(O)C[C@@H](c2nc(-c3nc([C@H]4C[C@@](C)(O)C4)n4ccnc(N)c34)c3c(N)nccn32)C1]

In other aspects, various resins were investigated to scavenge residual palladium from Suzuki coupling reaction streams. The solutions were treated at elevated temperature with resin. The resins were removed by filtration, washed with water, and the filtrates were concentrated in vacuo. The resins used in the study are shown in Table 1. The MTCf resin demonstrated strong activity along with the MP-TMT resin. Furthermore, the MTCf resin was utilized in conjunction with activated charcoal to further remove residual palladium and improve the purity.

TABLE 1

| Resin | Active Functional group |
|---|---|
| SPM32 | Thiol |
| SPM32f | Thiol |
| SEM26 | Thiol |
| STA3 | Triamine |
| SPM36 | Thiol |
| SPM36f | Thiol |
| SEA | Amine |
| MTCf | Thiourea |
| SCYT1 | N-acetyl cysteine |
| MP-TMT | Trimercaptotriazine |

The present invention further concerns the resin in (e), which can comprise at least one of MP-TMT, PL-TMT, MTCf, SPM32, SPM32f, SEA, SEM26, STA3, SPM36, SPM36f or SCYT1.

The present invention further concerns (e), which can comprise filtering said liquid through activated charcoal.

The present invention further concerns (e), which can comprise filtering said liquid through charcoal followed by treating said liquid with MTCf resin and/or MP-TMT resin.

The present invention further concerns the alcohol in (g), which can comprise 2-propanol, methanol, ethanol, n-propanol, n-butanol, sec-butanol, t-butanol, or iso-butanol.

The present invention further concerns the alcohol in (g), which can comprise 2-propanol.

The present invention further concerns the base in (h), which can comprise aqueous sodium hydroxide.

The present invention further concerns the pH in (c), which can be about pH 1-4 and a dimer impurity comprising Compound (8) that can be essentially completely removed.

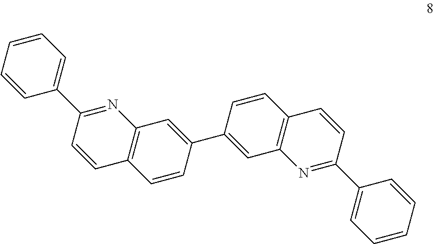 [SMILES: c1ccc(-c2ccc3ccc(-c4ccc5ccc(-c6ccccc6)nc5c4)cc3n2)cc1]

The present invention further concerns the pH in (c), which can be about pH 2.9 and a dimer impurity comprising Compound (8) that can be essentially completely removed.

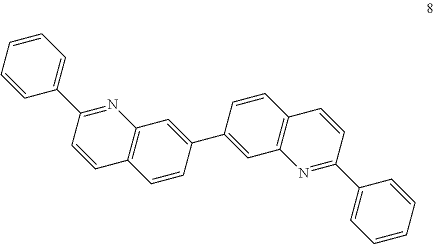 [SMILES: c1ccc(-c2ccc3ccc(-c4ccc5ccc(-c6ccccc6)nc5c4)cc3n2)cc1]

The present invention further concerns the pH in (h), which can be about pH 3-6 and a dimer impurity comprising Compound (9) that can be essentially completely removed.

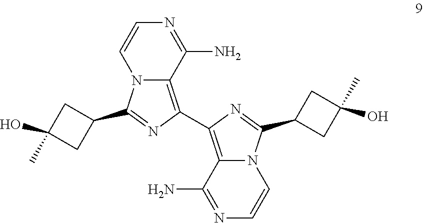 [SMILES: C[C@]1(O)C[C@@H](c2nc(-c3nc([C@H]4C[C@@](C)(O)C4)n4ccnc(N)c34)c3c(N)nccn32)C1]

The present invention further concerns the pH in (h), which can be about pH 5 and a dimer impurity comprising Compound (9) that can be effectively removed.

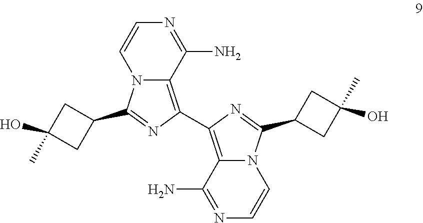 [SMILES: C[C@]1(O)C[C@@H](c2nc(-c3nc([C@H]4C[C@@](C)(O)C4)n4ccnc(N)c34)c3c(N)nccn32)C1]

The present invention further concerns the isolating precipitate in (i), which can comprise at least one of decanting solvent, evaporating solvent, or filtration, and in some embodiments, drying to the hydrate or hemi-hydrate.

The present invention further concerns the isolating precipitate in (i), which can comprise heating the precipitate in 2-propanol.

The present invention further concerns the isolating precipitate in (i), which can comprise washing the precipitate in 2-propanol and in some embodiments, drying the precipitate under vacuum.

The present invention further concerns the palladium catalyst, which can comprise palladium acetate and triphenylphosphine.

The present invention further concerns reaction (a), which can comprise heating to about 95° C. to 125° C.

The present invention further concerns the process, which can result in at least about 10 kg of OSI-906 having a purity of at least about 90%.

The present invention further concerns the process, which can result in at least about 20 kg of OSI-906 having a purity of at least about 90%.

The present invention further concerns the palladium removed in (e), which can result in an OSI-906 as a material containing less than 50 ppm, less than 20 ppm, or less than 10 ppm palladium.

The present invention further concerns the process, which can provide OSI-906 having a particle size distribution of about D90<70 μm and D50<40 μm, about D90<50 μm and D50<30 μm, about D90<40 μm and D50<15 μm, or about D90<20 μm and D50<10 μm.

The present invention further concerns the process having an overall yield of at least about 50% and providing OSI-906 with a purity of at least about 98%.

The present invention further concerns the above process having a yield of at least about 40%, 50%, or 65%.

The present invention further concerns the above process providing OSI-906 with a purity of at least about 80%, 90%, 95%, 98%, or 99%.

The present invention further concerns the isolating precipitate in (i), which can comprise:
(j) treating the precipitate with an acid in solution;
(k) heating the solution; and
(l) isolating an OSI-906 salt.

The present invention further concerns the acid in (j), which can comprise hydrochloric acid, L-tartaric acid, sulfuric acid, fumaric acid or methane sulfonic acid.

The invention includes the salts of OSI-906.

The present invention further concerns the heating the solution in (k), which can comprise heating to about 70° C. to 110° C.

The present invention further concerns Compound (2) in (a), which can be prepared by reacting Compound (4) with an amine to obtain at least about 1 kg of Compound (2).

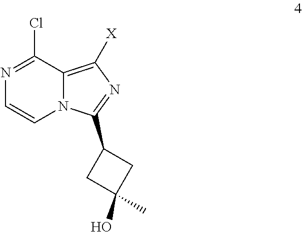

The present invention further concerns Compound (2) in (a), which can be prepared by (m) reacting Compound (4).

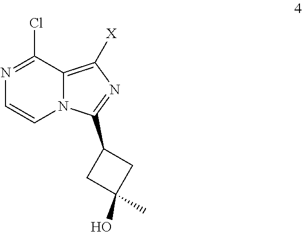

wherein X is Br, or I; and
with ammonia in a compatible solvent to obtain at least about 1 kg of Compound (2) with an overall yield of at least about 80%.

The present invention further concerns the ammonia, which can comprise an ammonia solution.

The present invention further concerns the process wherein X is Br.

The present invention further concerns the ammonia, which can comprise about a 35% ammonia solution, or about a 30% ammonia solution.

The present invention further concerns reaction (m), which can be carried out at about 100 psi or less, about 45-65 psi or less, or about 30 psi or less.

The present invention further concerns reaction (m), which can be carried out in 2-propanol, methanol, ethanol, isopropanol, n-propanol, n-butanol, sec-butanol, t-butanol or iso-butanol.

The present invention further concerns reaction (m), which can be carried out in 2-propanol.

The present invention further concerns reaction (m), which can comprise heating to about 65° C. to 95° C.

The present invention further concerns reaction (m), which can comprise concentrating volume of the solution followed by cooling to about −0° C. to −10° C.

The present invention further concerns the process, which results in at least about 10 kg of Compound (2), or at least about 20 kg of Compound (2).

The present invention further concerns the process having an overall yield of at least about 70% or more of Compound (2), at least about 80% or more of Compound (2), at least about 88% or more of Compound (2), or at least about 90% or more of Compound (2).

The present invention further concerns Compound (4), which can be prepared by reacting Compound (5) with a methylating reagent to obtain at least about 1 kg of Compound (4).

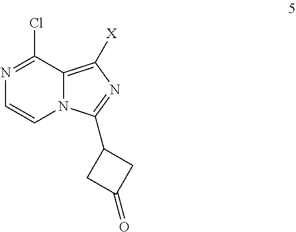

The present invention further concerns Compound (4) in (m), which can be prepared by (n) reacting Compound (5)

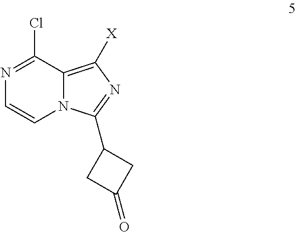

wherein X is Br, or I;
with a methylating reagent in a compatible organic solvent to obtain at least about 1 kg of Compound (4) with an overall yield of at least about 70%;
(o) quenching the reaction with an aqueous proton source;
(p) separating an aqueous phase from an organic phase;
(q) adjusting the pH of the aqueous phase to about pH 7-8 with an acid;
(r) extracting the Compound (4) from the aqueous phase with a solvent;
(s) combining the extract, washing the extract with an aqueous base and separating the extract;
(t) adding a solvent to the extract and concentrating the extract;
(u) heating the concentrated extract;
(v) cooling the concentrated extract; and
(w) isolating crystalline Compound (4).

The present invention further concerns the methylating reagent, which can comprise methyl magnesium bromide.

The present invention further concerns the pH in (q), which can be about 7-9 or 7-8.

The present invention further concerns the process wherein X is Br.

The present invention further concerns reaction (n), which can be carried out in THF.

The present invention further concerns reaction (n), which can comprise cooling to about −10° C. to −55° C., which can effectively prevent formation of a 7-methyl impurity Compound (10):

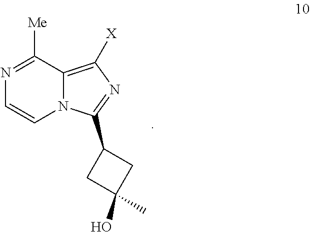

The present invention further concerns reaction (n), which can comprise cooling to about −55° C. to −65° C., which can effectively prevent formation of a 7-methyl impurity Compound (10).

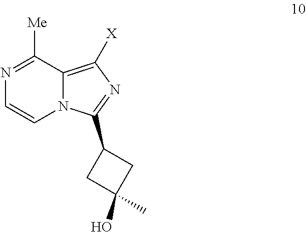

The present invention further concerns the proton source in (o), which can comprise ammonium chloride.

The present invention further concerns the solvent in (r), which can comprise t-butyl ether or ethyl ether.

The present invention further concerns the acid in (q), which can comprise about 1N HCl, or about 6N HCl.

The present invention further concerns the base in (s), which can comprise about 1N sodium hydroxide.

The present invention further concerns the base wash in (s), which can effectively remove a 7-hydroxy impurity Compound (11).

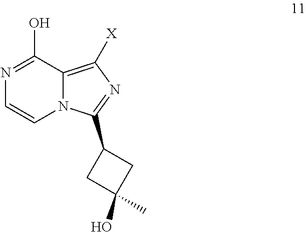

The present invention further concerns the solvent in (t), which can comprise toluene.

The present invention further concerns the isolating in (w), which can comprise crystallizing Compound (4) in toluene, and can remove an isomeric impurity.

The present invention further concerns the process, which can result in at least about 10 kg of Compound (4), or at least about 20 kg of Compound (4).

The present invention further concerns the process having a yield of at least about 60% of Compound (4), or at least about 70% of Compound (4), at least about 78% of Compound (4), or at least about 80% of Compound (4).

The invention includes pharmaceutical compositions comprising OSI-906 prepared according to the invention with or without a pharmaceutically acceptable carrier.

The present invention further concerns using the composition for the treatment of diseases including cancer.

In the process, bicyclic Compounds (5) (X is Cl, Br or I) can be assembled by the condensation of 2-aminomethyl-3-chloropyrazine with an activated aryl, heteroaryl, alkyl, or cycloalkyl carboxylic acids as disclosed and incorporated herein in US 2006/0235031 and US 2007/0129547.

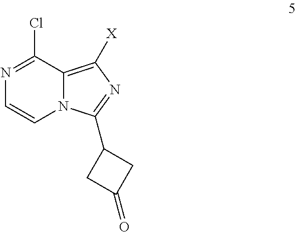

Compounds (6) (R¹ and R² are OH, alkoxy or R¹ and R² combine to form cyclic boronic ester) can be prepared as disclosed and incorporated herein in US 2006/0235031.

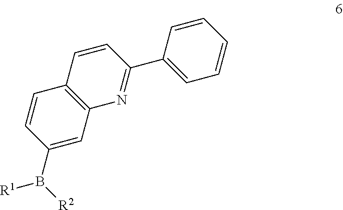

### EXAMPLES

### Example 1

### cis-3-[8-amino-1-(2-phenyl-quinolin-7-yl)-imidazo[1,5-a]pyrazin-3-yl]-1-methylcyclobutanol (OSI-906) (Compound 1)

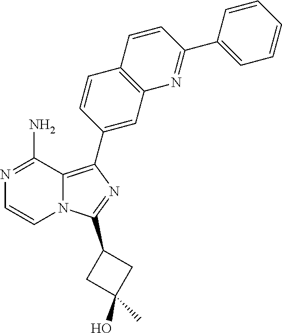 [SMILES: C[C@]1(O)C[C@@H](c2nc(-c3ccc4ccc(-c5ccccc5)nc4c3)c3c(N)nccn32)C1]

A vessel was charged with DMF (79 kg), cis-3-(8-amino-1-bromo-imidazo[1,5-a]pyrazin-3-yl)-1-methylcyclobutanol (16.725 kg), 2-phenyl-7-(4,4,5,5-tetramethyl-[1,3,2]dioxaborolan-2-yl)-quinoline (22.4 kg), triphenylphosphine (0.586 kg), cesium carbonate (36.7 kg) and water (20.1 kg). The reaction mixture was degassed and heated to 95-105° C. and a solution of palladium acetate (0.125 kg) in DMF (9.8 kg) was added and rinsed in with DMF (5.9 kg). After the reaction was complete, water (154 kg) was added keeping the temperature above 70° C. The resultant slurry was cooled and the solid was collected by filtration. After washing with a mixture of DMF (9.4 kg) and water (23.4 kg) and then water (67 kg) the solid was suspended in water (167 kg) at 50° C. and the pH of the mixture was adjusted to 2.9 with 6N hydrochloric acid (10.9 kg). The resultant yellow slurry was filtered to remove the major impurities and the cake was washed with water (67 kg). The acid solution was stirred at 50-55° C. and polymer bound trimercaptotriazine resin (MP-TMT) (4.9 kg) was added. The mixture was stirred for 23 hours, the resin was removed by filtration and the cake was washed with water (58 kg).

The resultant acid solution was diluted with 2-propanol (82 kg), the temperature was adjusted to 35-45° C. and the pH was adjusted to 5.0 by the addition of 1N sodium hydroxide solution. The mixture was cooled, the yellow product was collected by filtration and was washed with water (33 kg). The solid was re-suspended in water (157 kg) stirred, filtered and washed with water (125 kg). The solid was dried under vacuum at 45-55° C. (the resulting material was a hemihydrate of OSI-906 designated Form C) and was then stirred in refluxing 2-propanol (157 kg) for 3 hours. The mixture was cooled and the solid was isolated by filtration. After washing with 2-propanol (26.7 kg), the product was dried at 45-55° C. under vacuum to yield 15.6 kg (65% yield) of OSI-906. The resulting material was an anhydrous crystalline form of OSI-906 designated Form A.

### Example 2

### cis-3-(1-bromo-8-chloro-imidazo[1,5-a]pyrazin-3-yl)-1-methylcyclobutanol

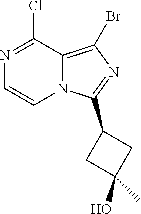 [SMILES: C[C@]1(O)C[C@@H](c2nc(Br)c3c(Cl)nccn32)C1]

THF (87 kg) and 3M methyl magnesium chloride (83.6 kg) were charged to a vessel. The contents were cooled to −65 to −55° C. and 3-(1-bromo-8-chloro-imidazo[1,5-a]pyrazin-3-yl)-cyclobutanone (33.0 kg) in THF (253 kg) was added, maintaining the temperature at −65° C. to −45° C.

The charged vessel was rinsed with THF (41 kg) and the reaction mixture was stirred at −65 to −45° C. until reaction completion. Preferably, the level of iron present in the reaction is about 100 ppm or less, or about 20 ppm or less. These conditions are suitable to achieve the desired stereoselectivity. A 5% ammonium chloride solution (462 kg) was added slowly while maintaining the temperature below 10° C. The aqueous layer was then separated, the pH was adjusted to pH 7-8 by the addition of 6N hydrochloric acid and the mixture was extracted with methyl t-butyl ether (2×145 kg). The combined organic extracts were washed sequentially with 1N sodium hydroxide solution (330 kg) and 20% sodium chloride solution (2×330 kg). THF (767 kg) was then added and the solution was distilled to a residual volume of 165 L. Toluene (567 kg) was added and again the mixture was distilled to a volume of 165 L. The mixture was heated to 85-90° C. until complete dissolution was achieved and then cooled to 20-30° C. to crystallize the product. The solids were collected by filtration, washed with toluene (2×41 kg) and dried at 50-60° C. under vacuum. Yield was 78%. ¹H NMR (300 MHz, DMSO-d₆) δ 8.3 (d, 1H), 7.4 (d, 1H), 5.2 (s, 1H), 3.5 (m, 1H), 2.4 (m, 4H), 1.4 (s, 3H).

### Example 3

### cis-3-(8-amino-1-bromo-imidazo[1,5-a]pyrazin-3-yl)-1-methylcyclobutanol

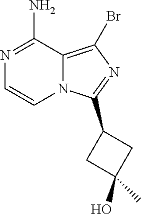 [SMILES: C[C@]1(O)C[C@@H](c2nc(Br)c3c(N)nccn32)C1]

Cis-3-(1-bromo-8-chloro-imidazo[1,5-a]pyrazin-3-yl)-1-methylcyclobutanol (27.1 kg), isopropanol (65 kg) and 30% ammonia solution (165 kg) were charged to a suitable vessel. The vessel was sealed and the mixture was heated and stirred for 18 hours at 75 to 85° C. and then cooled. The vessel was vented to a scrubber and water (22 kg) was added. The mixture was concentrated under vacuum to a residual volume of 73-89 L and was then cooled to <5° C. The product was collected by filtration and washed with water (2×108 kg). The product was dried at 40-50° C. under vacuum. Yield was 88%. ¹H NMR (300 MHz, DMSO-d₆) δ 7.5 (d, 1H), 7.0 (d, 1H), 6.6 (br s, 2H), 5.2 (s, 1H), 3.4 (m, 1H), 2.4 (m, 4H), 1.4 (s, 3H).

### Example 4

### cis-8-amino-3-(3-hydroxy-3-methyl-cyclobutyl)-1-(2-phenyl-quinolin-7-yl)-imidazo[1,5-a]pyrazin-7-ium chloride

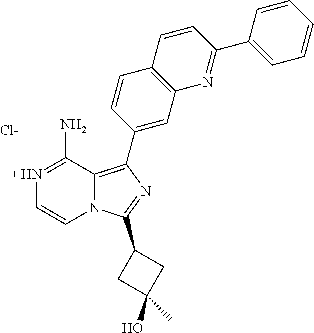 [SMILES: [Cl-] ; C[C@]1(O)C[C@@H](c2nc(-c3ccc4ccc(-c5ccccc5)nc4c3)c3c(N)nccn32)C1]

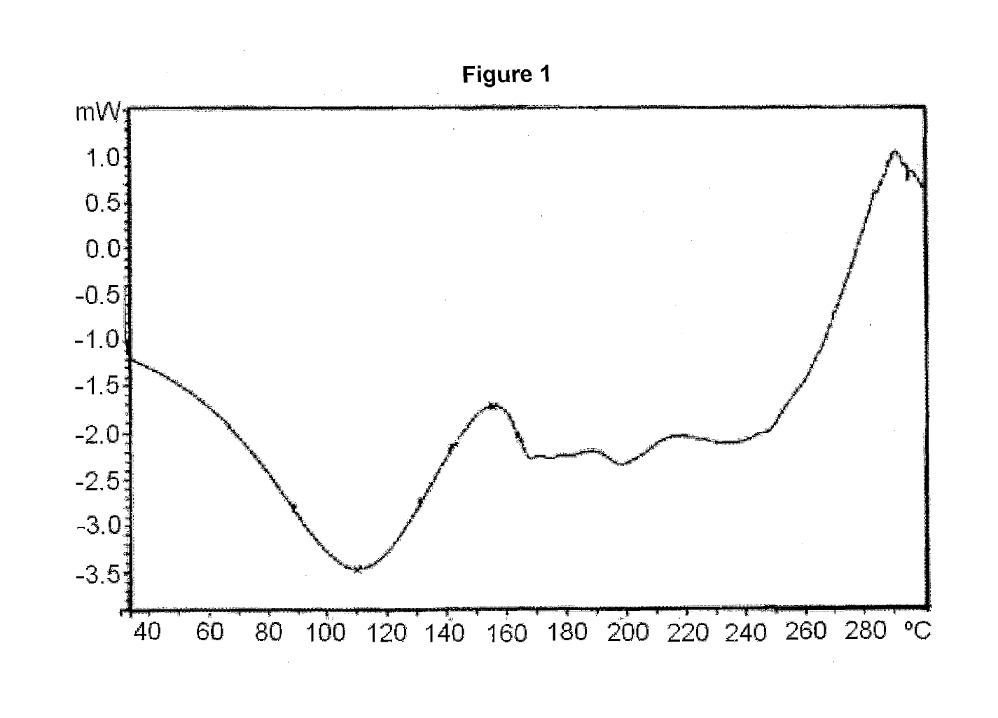

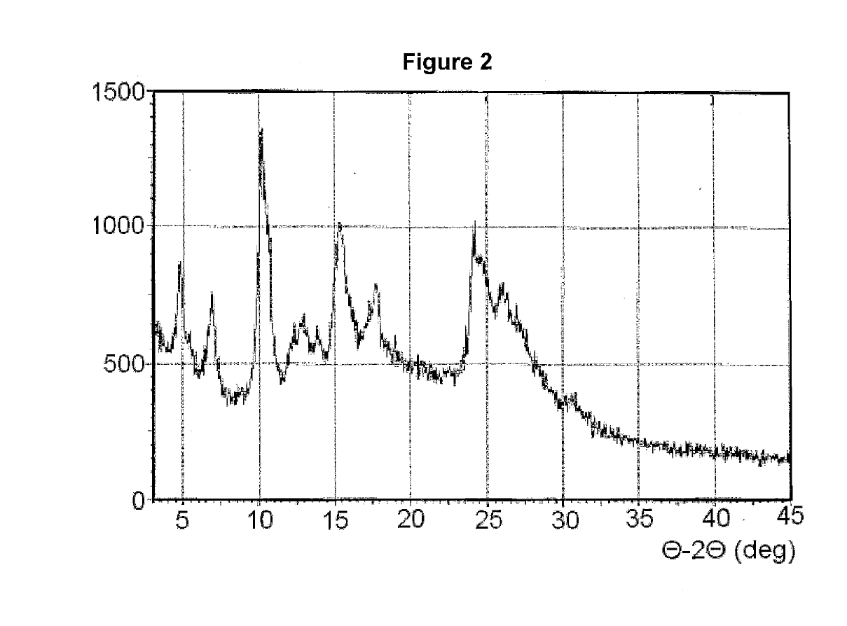

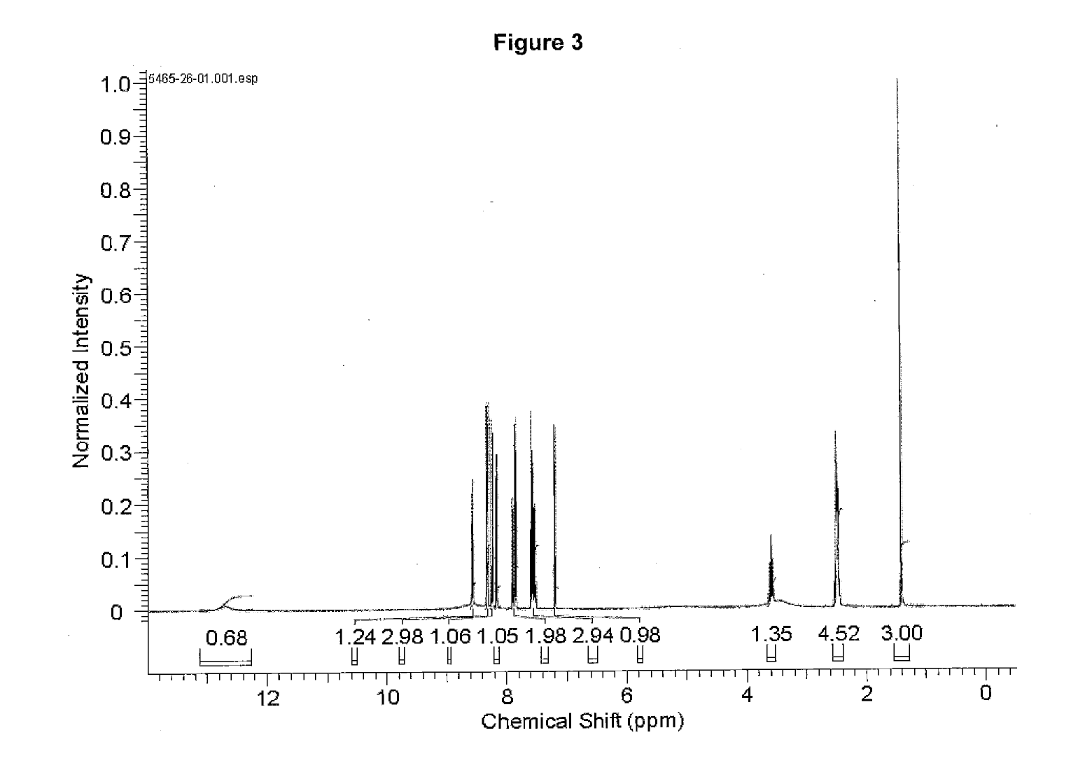

This material was prepared by heating OSI-906 with an equivalent of hydrochloric acid in water and then allowing the solution to cool. The solid was filtered from the cooled mixture and dried. The XRPD and DSC suggest a semi-crystalline material. The DSC, XRPD, and ¹H NMR (300 MHz, DMSO-d₆) of the sample were recorded and are reproduced in FIGS. 1, 2, and 3, respectively.

### Example 5

### cis-8-amino-3-(3-hydroxy-3-methyl-cyclobutyl)-1-(2-phenyl-quinolin-7-yl)-imidazo[1,5-a]pyrazin-7-ium3-carboxy-2,3-dihydroxy-propionate

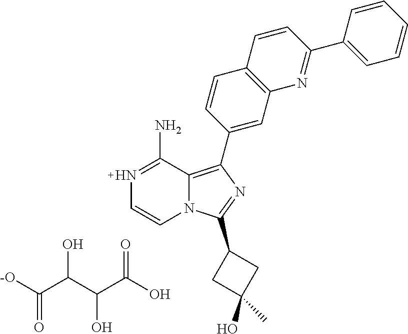

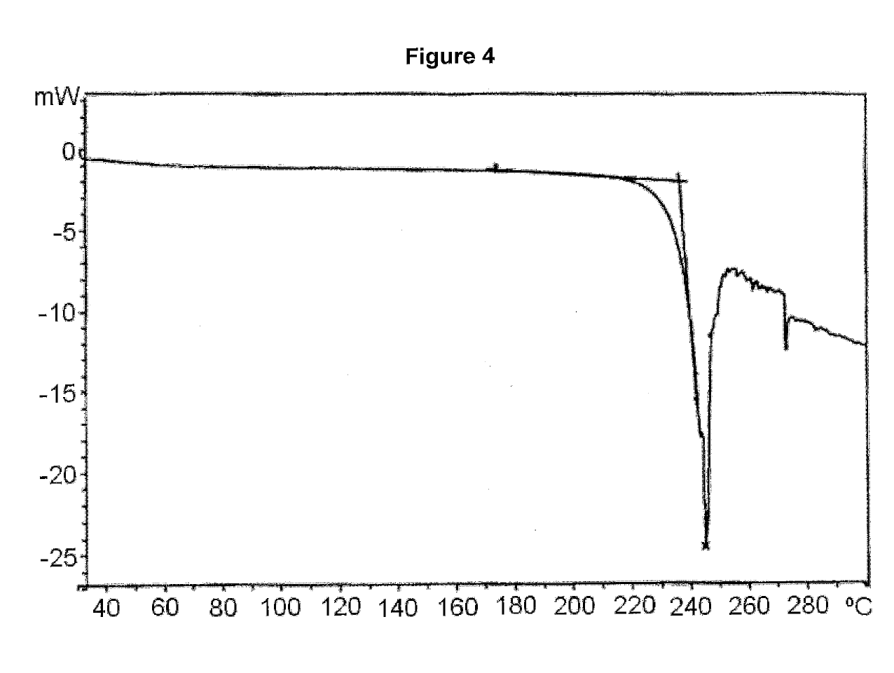

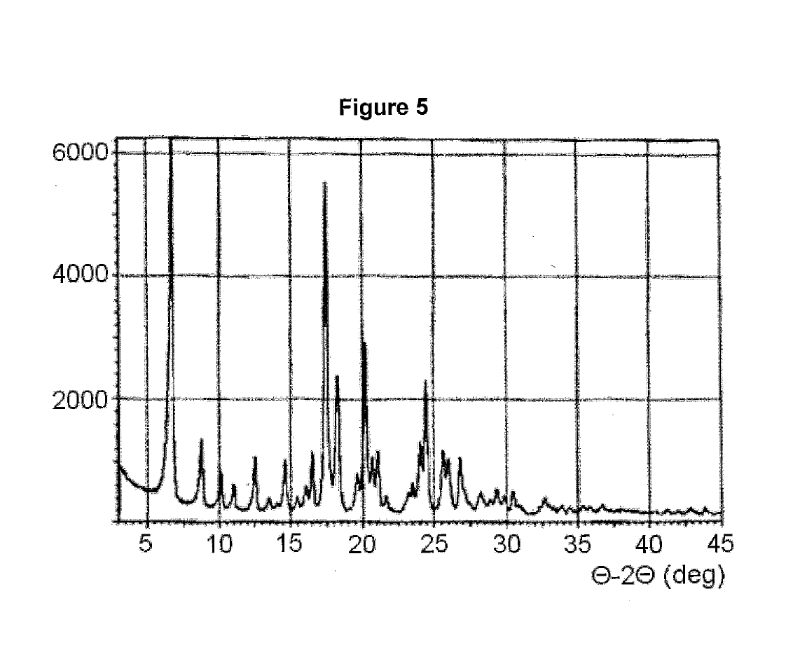

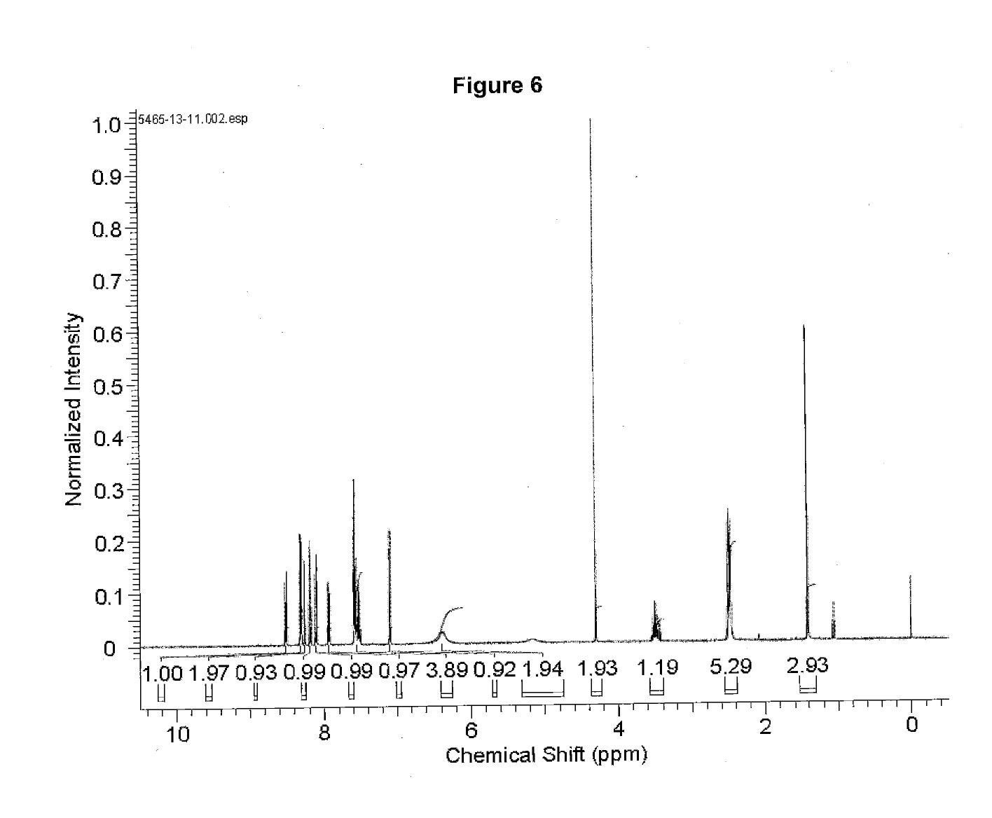

This material was prepared by heating OSI-906 with a slight excess of L-tartaric acid in ethanol and then allowing the mixture to cool. The solid was collected by filtration and dried. The DSC, XRPD, and ¹H NMR (300 MHz, DMSO-d₆) of the sample were recorded and are reproduced in FIGS. 4, 5, and 6, respectively.

### Example 6

### cis-8-amino-3-(3-hydroxy-3-methyl-cyclobutyl)-1-(2-phenyl-quinolin-7-yl)-imidazo[1,5-a]pyrazin-7-ium hydrogen sulfate

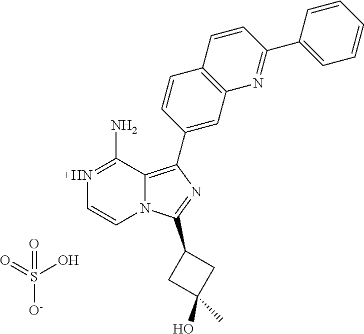 [SMILES: O=S(=O)([O-])O ; C[C@]1(O)C[C@@H](c2nc(-c3ccc4ccc(-c5ccccc5)nc4c3)c3c(N)nccn32)C1]

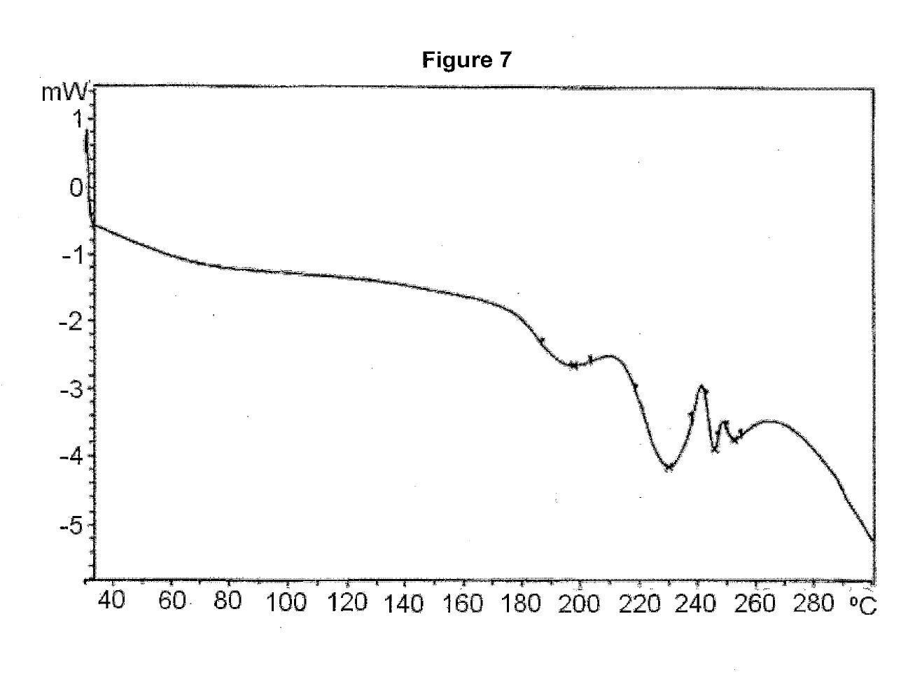

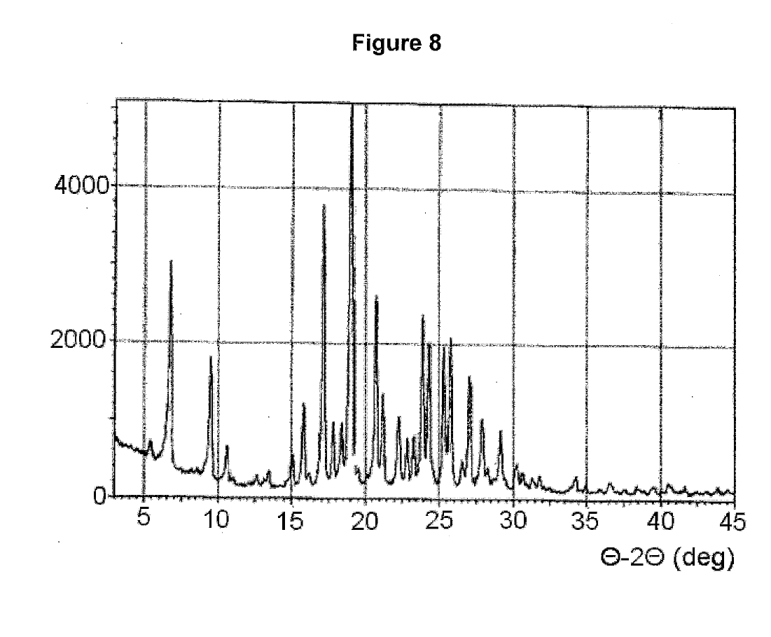

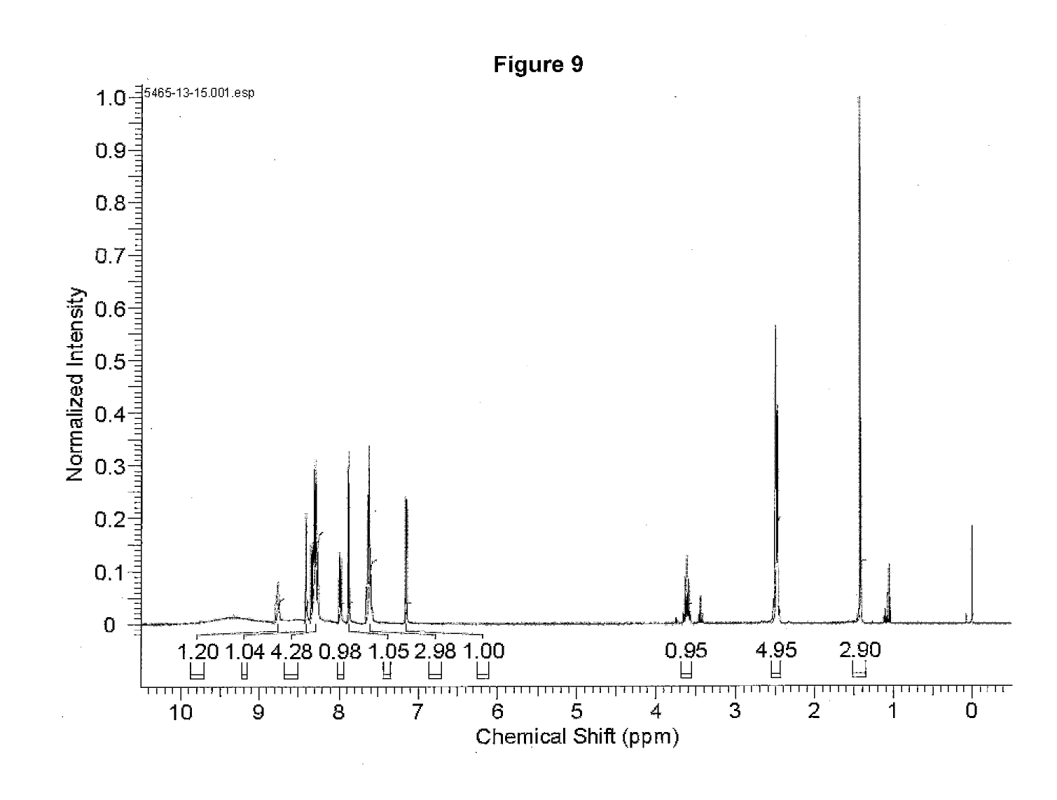

This material was prepared by heating OSI-906 with a slight excess of sulfuric acid in ethanol and then allowing the mixture to cool. The solid was collected by filtration and dried. The DSC, XRPD, and ¹H NMR (300 MHz, DMSO-d₆) of the sample were recorded and are reproduced in FIGS. 7, 8, and 9, respectively.

### Example 7

### cis-8-amino-3-(3-hydroxy-3-methyl-cyclobutyl)-1-(2-phenyl-quinolin-7-yl)-imidazo[1,5-a]pyrazin-7-ium3-carboxy-acrylate

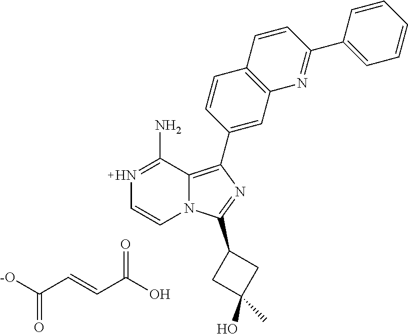

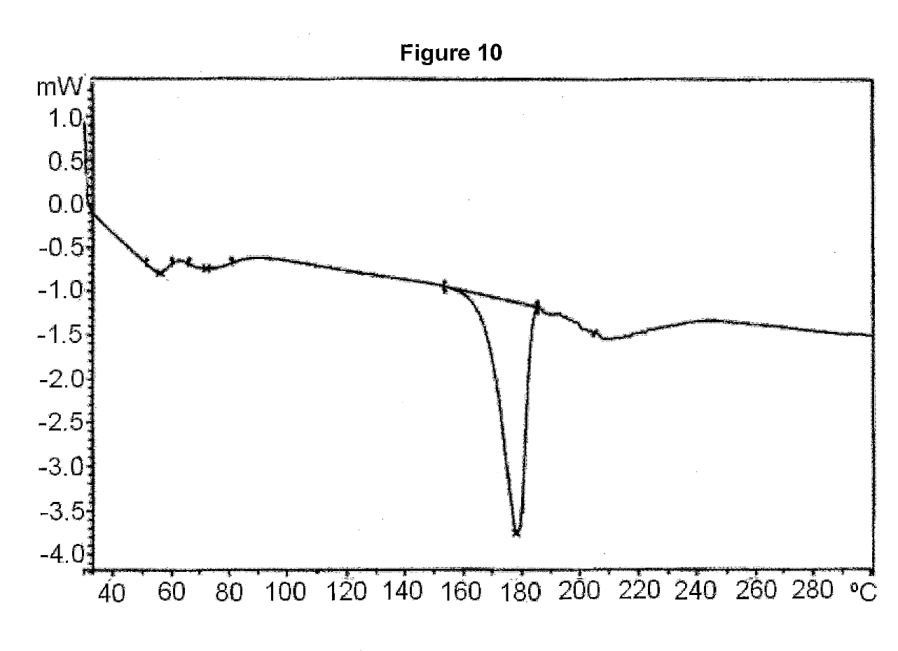

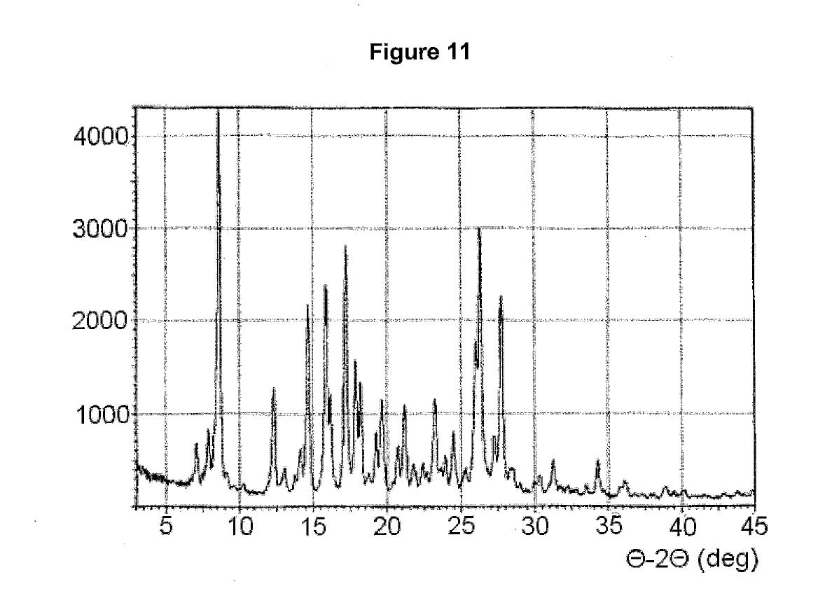

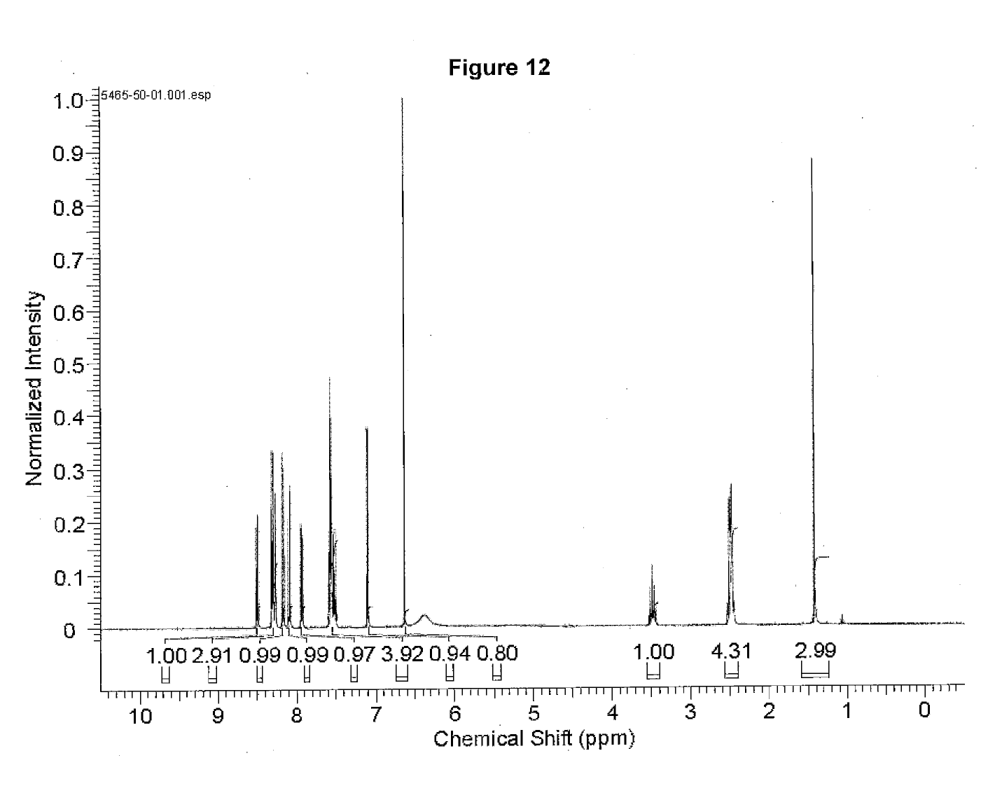

This material was prepared by heating OSI-906 with a slight excess of fumaric acid in ethanol/water and then allowing the mixture to cool. The solid was collected by filtration and dried. The DSC, XRPD, and ¹H NMR (300 MHz, DMSO-d₆) of the sample were recorded and are reproduced in FIGS. 10, 11, and 12, respectively.

### Example 8

### cis-8-amino-3-(3-hydroxy-3-methyl-cyclobutyl)-1-(2-phenyl-quinolin-7-yl)-imidazo[1,5-a]pyrazin-7-ium methanesulfonate

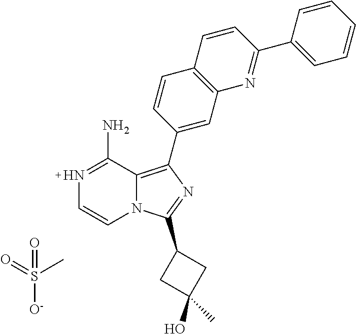

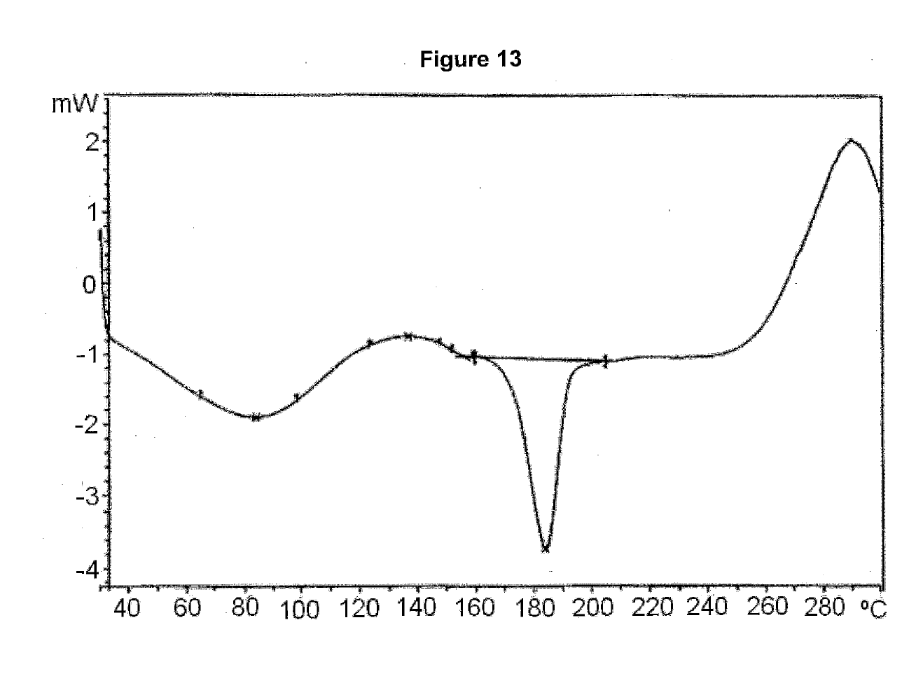

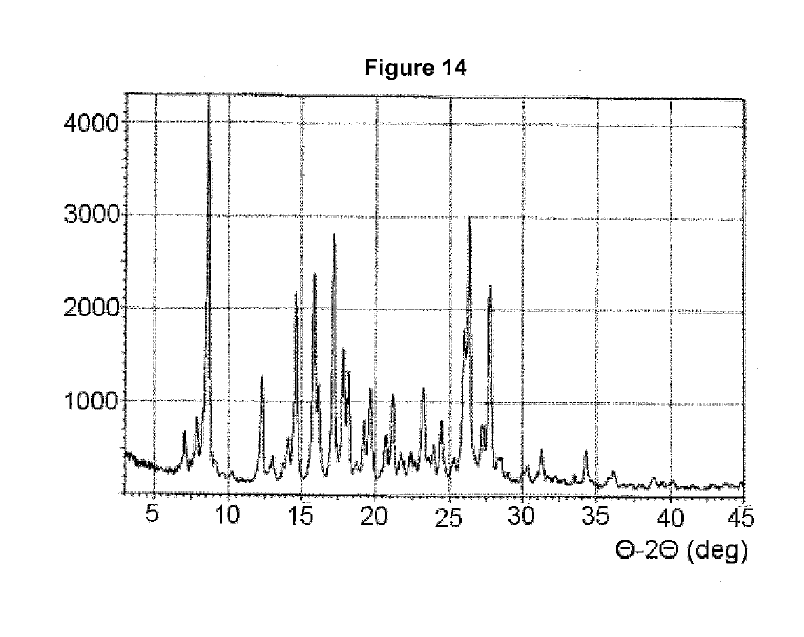

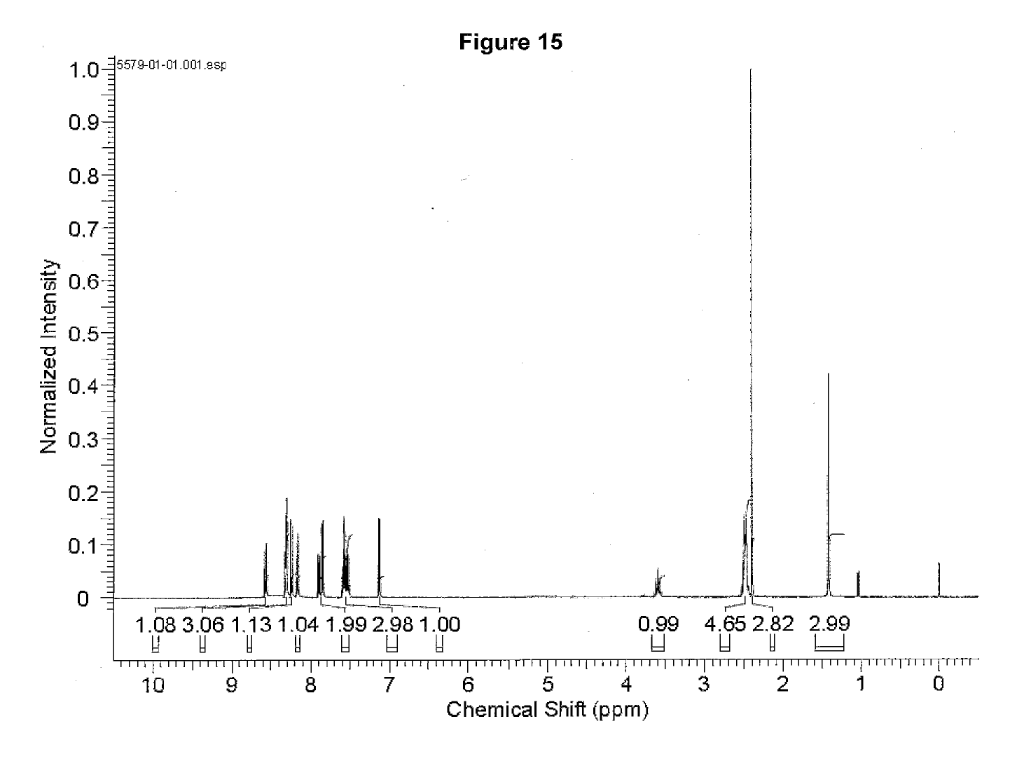

This material was prepared by heating OSI-906 with a slight excess of methane sulfonic acid in 2-propanol and then allowing the mixture to cool. The solid was collected by filtration and dried. The DSC, XRPD, and ¹H NMR (300 MHz, DMSO-d₆) of the sample were recorded and are reproduced in FIGS. 13, 14, and 15, respectively.

### GENERAL DEFINITIONS AND ABBREVIATIONS

Unless otherwise specified, terms used herein shall have the broadest meanings as commonly understood by one of ordinary skill in the art. Each variable definition above includes any subset thereof.

The term “isolating” refers to indicate separation or collection or recovery of the compound of the invention being isolated in the specified form.

The term “active agent” of the invention means a compound of the invention in any salt, polymorph, crystal, solvate, or hydrated form.

The term “pharmaceutically acceptable salt(s)” is known in the art and includes salts of acidic or basic groups which can be present in the compounds and prepared or resulting from pharmaceutically acceptable bases or acids.

In descriptions and claims where subject matter (e.g., substitution at a given molecular position) is recited as being selected from a group of possibilities, the recitation is specifically intended to include any subset of the recited group. In the case of multiple variable positions or substituents, any combination of group or variable subsets is also contemplated.

The term “aliphatic” means any hydrocarbon moiety, and can contain linear, branched, and cyclic parts, and can be saturated or unsaturated. The term “alkyl” means any saturated hydrocarbon group that is straight-chain or branched. Examples of alkyl groups include methyl, ethyl, propyl, 2-propyl, n-butyl, iso-butyl, tert-butyl, pentyl, and the like.

The term “pharmaceutical composition” means an active compound in any form suitable for effective administration to a subject, e.g., a mixture of the compound and at least one pharmaceutically acceptable carrier.

As used herein, a “physiologically/pharmaceutically acceptable carrier” means a carrier or diluent that does not cause significant irritation to an organism and does not abrogate the biological activity and properties of the administered compound.

A “pharmaceutically acceptable excipient” means an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

The following abbreviations are used:

min. minute(s)

h hour(s)

d day(s)

RT or rt room temperature

t_{R} retention time

L liter

mL milliliter

mmol millimole

μmol micromole

equiv. or eq. equivalent

NMR nuclear magnetic resonance

LC/MS liquid chromatography mass spectrometry

HPLC high performance liquid chromatography

TLC thin layer chromatography

CDCl₃ deuterated chloroform

CD₃OD or MeoD deuterated methanol

DMSO-d₆ deuterated dimethylsulfoxide

DCM dichloromethane

THF THF

EtOAc ethyl acetate

MeCN acetonitrile

DMSO dimethylsulfoxide

DME 1,2-dimethoxyethane

DMF N,N-dimethylformamide

DIPEA diisopropylethylamine

DMAP 4-dimethylaminopyridine

IPA isopropanol

1. A process for preparing OSI-906 or a pharmaceutically acceptable salt thereof comprising:
(a) reacting Compounds 2 and 6: 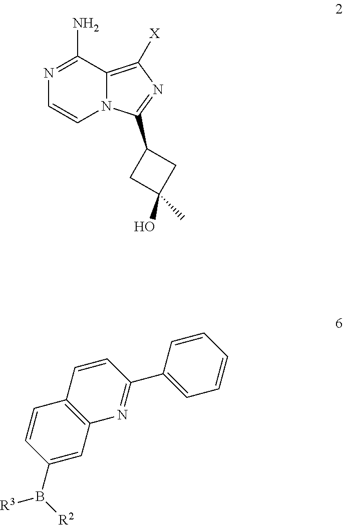
wherein X is Cl, Br, or I; R² and R³ are independently OH or OR⁴; or R² and R³ combine to form a cyclic boronic ester; and R⁴ is C₁-C₆aliphatic;
under palladium catalyst mediated coupling conditions;
(b) diluting with water and collecting solids;
(c) suspending the solids in water and adjusting the pH to about 2-3 with a suitable acid to provide a dissolved salt form of OSI-906;
(d) separating remaining solids from liquid;
(e) mixing the liquid from (d) with a suitable metal scavenging resin to remove palladium;
(f) removing the resin;
(g) diluting the liquid with a suitable alcohol;
(h) adjusting the pH with base to about 5-6 to precipitate the OSI-906;
(i) isolating the precipitate from (h); and
thereby obtaining at least about 1 kg of OSI-906 having a purity of at least about 90% without further purification.

2-4. (canceled)

5. The process of claim 1, wherein (e) further comprises filtering the liquid through activated charcoal.

6. The process of claim 5, wherein (e) further comprises treating the liquid with MTCf resin and/or MP-TMT resin.

7-8. (canceled)

9. The process of claim 1, wherein the pH in (c) is about pH 2.9 and any Compound (8) present is substantially all removed

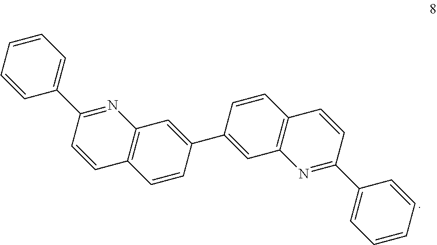 [SMILES: c1ccc(-c2ccc3ccc(-c4ccc5ccc(-c6ccccc6)nc5c4)cc3n2)cc1]

10. The process of claim 1, wherein pH in (h) is about pH 5 and any Compound (9) present is substantially all removed

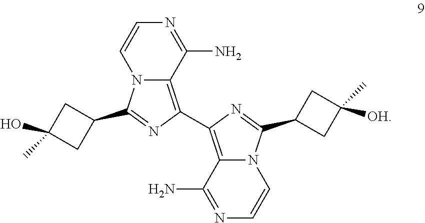 [SMILES: C[C@]1(O)C[C@@H](c2nc(-c3nc([C@H]4C[C@@](C)(O)C4)n4ccnc(N)c34)c3c(N)nccn32)C1]

11-15. (canceled)

16. The process of claim 1, which results in at least about 10 kilograms of OSI-906 having a purity of at least about 90%.

17. The process of claim 1, having a yield of at least about 50% and providing OSI-906 as a material having a purity of at least about 98%.

18. The process of claim 1, which produces OSI-906 as a material containing less than 20 ppm palladium.

19. The process of claim 1, which provides OSI-906 having a particle size distribution of about D90<40 μm and about D50<15 μm.

20. The process of claim 1, wherein the isolating precipitate in (i) further comprises:
(j) treating the precipitate with an acid in solution;
(k) heating the solution; and
(l) isolating an OSI-906 salt.

21-22. (canceled)

23. The process of claim 1, wherein the Compound (2) in (a) is prepared by (m) reacting Compound (4) 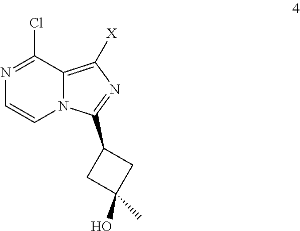
wherein X is Br, or I;
with an amine in a compatible solvent to obtain at least about 1 kg of Compound (2) in a yield of at least about 80%.

24-26. (canceled)

27. The process of claim 23, wherein reaction (m) is carried out at about 45-65 psi.

28-31. (canceled)

32. The process of claim 23, having a yield of at least about 88% or more of Compound (2).

33. The process of claim 23, wherein the Compound (4) in (m) is prepared by (n) reacting Compound (5) 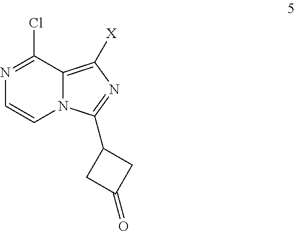
wherein X is Br, or I;
with a methylating reagent in a compatible organic solvent to obtain at least about 1 kg of Compound (4) with a yield of at least about 70%;
(o) quenching the reaction with an aqueous proton source;
(p) separating an aqueous phase from an organic phase;
(q) adjusting the pH of the aqueous phase to about pH 7-8 with an acid;
(r) extracting the Compound (4) from the aqueous phase with a solvent;
(s) combining the extract, washing the extract with an aqueous base and separating the extract;
(t) adding a solvent to the extract and concentrating the extract;
(u) heating the concentrated extract;
(v) cooling the concentrated extract; and
(w) isolating crystalline Compound (4).

34-36. (canceled)

37. The process of claim 33, wherein reaction (n) comprises cooling to about −55° C. to −65° C., and wherein the resulting Compound 5 is essentially free of Compound (10):

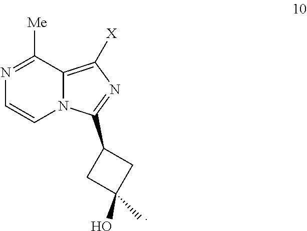

38-41. (canceled)

42. The process of claim 33, wherein the base wash in (s) effectively removes essentially any present Compound (11):

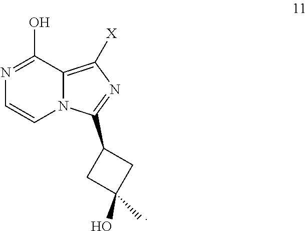

43-47. (canceled)
